(19) United States
(12) Patent Application Publication
(10) Pub. No.: US 2018/0133495 A1
(43) Pub. Date: May 17, 2018

(54) SYSTEM AND METHODS FOR STORING AN AUTOMATED EXTERNAL DEFIBRILLATOR

(71) Applicants: Alan Himelfarb, Avon, CO (US); Lynn Blake, Avon, CO (US)

(72) Inventors: Alan Himelfarb, Avon, CO (US); Lynn Blake, Avon, CO (US)

(21) Appl. No.: 15/354,542

(22) Filed: Nov. 17, 2016

(51) Int. Cl. A61N 1/39; B65D 81/18

(52) U.S. Cl. CPC A61N 1/3975; B65D 81/18

(57) ABSTRACT

A system is described for storing an automated external defibrillator (AED). The system may include an enclosure including a first portion to store the AED, and a second portion to store a battery to power a heating element. The system may further include a thermostat to monitor a temperature of the enclosure, the thermostat contained within the second portion of the enclosure. In some embodiments, the heating element may be configured to heat the first portion of the enclosure, and the heating element may be in electrical communication with the thermostat and may be contained within the first portion of the enclosure. The system may also include a controller configured to activate and deactivate power to the heating element, where the controller may be in electrical communication with the battery and the heating element, and may be contained within the second portion of the enclosure.

### BACKGROUND

The present invention relates generally to an automated external defibrillator (AED), and more particularly to a system for storing and maintaining the AED within an operable temperature range.

The American Heart Association (AHA) reports that nearly 300,000 people in the United States die every year from sudden cardiac arrest (SCA). Additionally, each year in the United States, there are approximately 359,400 Emergency Medical Services-assessed cardiac arrests outside of a hospital setting and on average, less than 10% of these cardiac arrest victims survive.

Early defibrillation, along with cardiopulmonary resuscitation (CPR), is the only way to restore an SCA victim's heart rhythm to a normal rate in many cases, and immediate CPR and early defibrillation may more than double a victim's chance of survival. In 2013, the AHA reported that 23% of out-of-hospital cardiac arrests were “shockable” arrhythmias, meaning cardiac arrests that would respond to a shock from an AED. Sadly, 64% of Americans do not have ready access to, and in some cases have never even seen, an AED Communities with AED programs have achieved survival rates of nearly 40% for cardiac arrest victims. For every minute that passes without CPR and defibrillation, the chances of survival decrease by 7-10%. Yet, across the Unites States there is a lack of available AEDs and persons trained to use AEDs. The moments between when a victim goes into SCA and when treatment is started are critical. Thus, it is imperative that AEDs be available in easily accessible locations. Yet, AEDs are rarely found outside, at least due to limitations of AED battery operability at low and below-freezing temperatures. If AEDs were to become available in outdoor locations, SCA survival rates would likely increase at least due to decreasing the time needed to locate an AED and/or for a first responder to administer aid.

Currently, AEDs may only be operable when kept above freezing temperatures, which may markedly limit the potential locations for AEDs stored outdoors. A system and method to allow AEDs to remain operable in below-freezing temperatures may therefore be desirable, and in many situations, may be lifesaving.

### SUMMARY

Typical batteries used for AED systems, such as lithium manganese dioxide, lithium-ion, or lithium-ion rechargeable batteries, may be optimally operable at warmer temperatures, and may lose functionality and charge at lower temperatures. For example, a battery that provides 100% capacity at 80 degrees Fahrenheit may only deliver partial capacity at lower temperatures. This lowered capacity may limit or entirely eliminate operability of an AED system relying on the battery for power. Furthermore, for batteries that are partially discharged, there is a heightened risk that the battery may freeze, thereby rendering the battery, and by extension the AED, entirely inoperable. Where AED systems are relied upon to save lives in situations where every minute counts, encountering an inoperable AED system may represent a significant risk to user survival.

A requirement that the battery powering the AED be stored in above-freezing temperatures may limit the availability of AED systems in outdoor locations susceptible to colder temperatures. A need for AED systems capable of being stored and maintaining operability in below-freezing temperatures exists. This may be particularly useful in colder climates, ski areas, and the like. Accordingly, systems and methods are described herein for storing an automated external defibrillator (AED). In one example, the system may include an enclosure including a first portion to store the AED, and a second portion to store a battery to power a heating element. The system may further include a thermostat to monitor a temperature of the enclosure, the thermostat contained within the second portion of the enclosure. In some embodiments, the heating element may be configured to heat the first portion of the enclosure, and the heating element may be in electrical communication with the thermostat and may be contained within the first portion of the enclosure. The system may also include a controller configured to activate and deactivate power to the heating element, where the controller may be in electrical communication with the battery and the heating element, and may be contained within the second portion of the enclosure.

In some examples, the system may further include a solar panel in electrical communication with the battery. In some examples, the solar panel may be positioned remotely from the enclosure containing the AED. In some examples, the solar panel may be positioned atop a structure, and the enclosure containing the AED may be affixed to a base of the structure. In some examples, the structure may be any of a telephone pole, a sign, a fence post, or a light post, or a combination thereof.

In some examples, the enclosure may be constructed of a rigid, waterproof material.

In some examples, the battery may be a 12-volt lead-acid battery.

In some examples, the enclosure may further include a global positioning system tracker or tamper resistant features, or a combination thereof.

In some examples, the controller may be operable to activate power to the heating element when the temperature of the second portion of the enclosure falls below 32 degrees Fahrenheit.

The present disclosure also relates to an enclosure for an automated external defibrillator (AED). In some examples, the enclosure may include a first portion of the enclosure configured for storing the AED. In some examples, the first portion of the enclosure may include means for heating the first portion of the enclosure. In some examples, the enclosure may also include a second portion of the enclosure separate from the first portion and configured for storing a battery to power the means for heating the first portion of the enclosure. In some examples, the second portion of the enclosure may include means for monitoring a temperature of the second portion of the enclosure. The second portion of the enclosure may also include means for determining that the temperature of the second portion of the enclosure is below a first threshold. The second portion of the enclosure may also include means for initiating the means for heating based at least in part on determining that the temperature of the second portion of the enclosure is below the first threshold.

In some examples, the means for determining that the temperature of the second portion of the enclosure is below the first threshold may further include means for determining that the temperature of the second portion of the enclosure is above a second threshold.

In some examples, the means for initiating the means for heating may include means for deactivating the means for heating based at least in part on determining that the temperature of the second portion of the enclosure is above the second threshold.

In some examples, the enclosure may further include means for powering the means for initiating heating of the enclosure. In some examples, the means for powering the means for initiating heating of the enclosure may be configured to rotate or separate from the enclosure, or a combination thereof. In some examples, the means for powering the means for initiating the heating of the enclosure may be positioned atop a structure, and the enclosure for the AED may be positioned at a base of the structure. In some examples, the structure may be at least one of a telephone pole, a sign, a fence post, or a light post, or any combination thereof.

In some examples, the enclosure may be configured to be portable, or waterproof or a combination thereof. In some examples, the enclosure may include means for tracking the position of the enclosure or providing tamper resistant features on the enclosure, or a combination thereof.

In some examples, the first threshold may be 32 degrees Fahrenheit.

The present disclosure also relates to a system for heating a battery to power an automated external defibrillator (AED). In some examples, the system may include an enclosure including a first portion to store the AED, and a second portion to store a battery to power a heating element. In some examples, the first portion may include a mounting bracket to support the AED. In some examples, the system may further include a thermostat to monitor a temperature of the second portion of the enclosure, where the thermostat may be contained within the second portion. In some examples, the heating element may be configured to heat the first portion of the enclosure, and may be in electrical communication with the thermostat, and may further be contained within the first portion of the enclosure. In some examples, the system may further include a controller in electrical communication with the battery to power the heating element and the heating element. In some examples, the controller may be contained within the second portion of the enclosure and may be operable to activate power to the heating element when the monitored temperature of the second portion drops below 32 degrees Fahrenheit. The controller may be further configured to deactivate power to the heating element when the monitored temperature of the enclosure increases above a threshold. In some examples, the system may further include a solar panel to charge the battery to power the heating element. In some examples, the solar panel may be affixed to an exterior portion of the enclosure and may be in electrical communication with the battery to power the heating element.

In some examples, the solar panel may be positioned remotely from the enclosure including the AED.

The foregoing has outlined broadly the features and technical advantages of examples according to this disclosure so that the following detailed description may be better understood. Additional features and advantages will be described below. The conception and specific examples disclosed may be readily utilized as a basis for modifying or designing other structures for carrying out the same purposes of the present disclosure. Such equivalent constructions do not depart from the scope of the appended claims. Characteristics of the concepts disclosed herein—including their organization and method of operation—together with associated advantages will be better understood from the following description when considered in connection with the accompanying figures. Each of the figures is provided for the purpose of illustration and description only, and not as a definition of the limits of the claims.

### BRIEF DESCRIPTION OF THE DRAWINGS

A further understanding of the nature and advantages of the present invention may be realized by reference to the following drawings. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 is a block diagram illustrating one example of an apparatus according to various embodiments of the invention;

FIG. 2 is a block diagram illustrating one example of an apparatus according to various embodiments of the invention; and

FIG. 3 is a block diagram illustrating an interior view of one example of an apparatus according to various embodiments of the invention.

While the embodiments described herein are susceptible to various modifications and alternative forms, specific embodiments have been shown by way of examples in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION OF THE INVENTION

This description provides examples, and is not intended to limit the scope, applicability or configuration of the invention. Rather, the ensuing description will provide those skilled in the art with an enabling description for implementing embodiments of the invention. Various changes may be made in the function and arrangement of elements.

Thus, various embodiments may omit, substitute, or add various procedures or components as appropriate. For instance, it should be appreciated that the methods may be performed in an order different than that described, and that various steps may be added, omitted or combined. Also, aspects and elements described with respect to certain embodiments may be combined in various other embodiments. It should also be appreciated that the following systems, methods, devices, and software may individually or collectively be components of a larger system, wherein other procedures may take precedence over or otherwise modify their application.

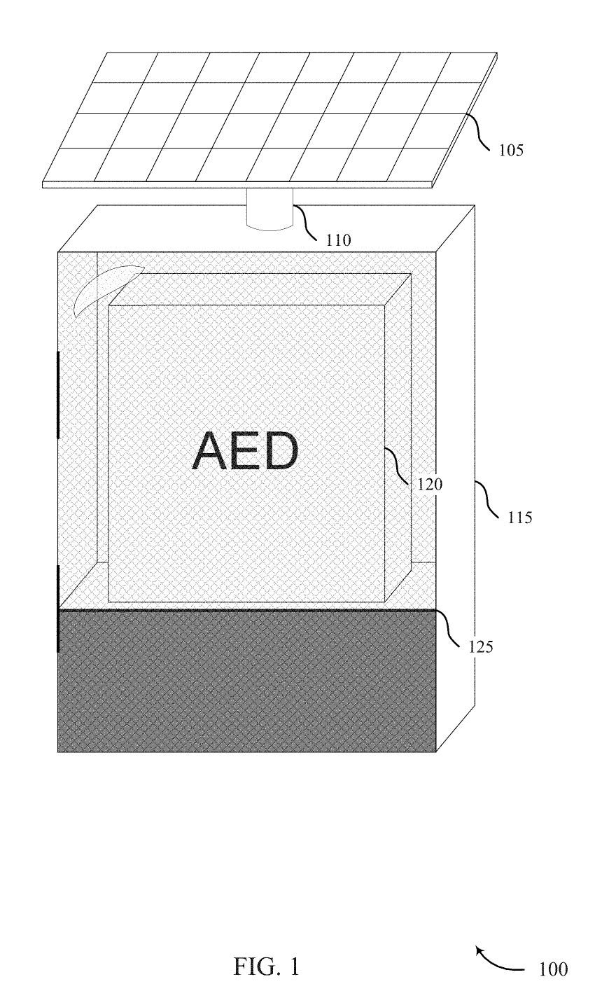

FIG. 1 is an example of an AED enclosure system 100 in accordance with various aspects of the disclosure. In some embodiments, the AED enclosure system 100 may include any of a solar panel 105, a mount 110 to connect the solar panel 105 to an enclosure 115, an AED 120 positioned within the enclosure 115, and a shelf 125 positioned within the enclosure 115. In the embodiment of system 100 illustrated, the AED enclosure 115 may take a rectangular or square shape, though other enclosure shapes are envisioned. The enclosure 115 may include a door or otherwise rotatable and/or removable front face, configured to allow for easy opening and closing of the enclosure 115 by a user needing urgent medical help. For example, the enclosure may include a door mounted to the front of the enclosure using hinges or any other suitable means to open and close the door. Within the enclosure 115, an AED 120 may be positioned on a shelf 125. The AED 120 may be any standard AED equipment that is self-contained and capable of delivering on-site defibrillation to a user experiencing sudden cardiac arrest.

The enclosure 115 may be constructed of a rigid, waterproof material, to enable the enclosure 115 to be stored in an outdoor, exposed area for extended periods of time, including year-round. The enclosure 115 may be coupled to the exterior of a building or other structure, may be placed or coupled with the ground or some grounded structure, or any other embodiment enabling the enclosure 115 to be positioned in an easily accessible outdoor area.

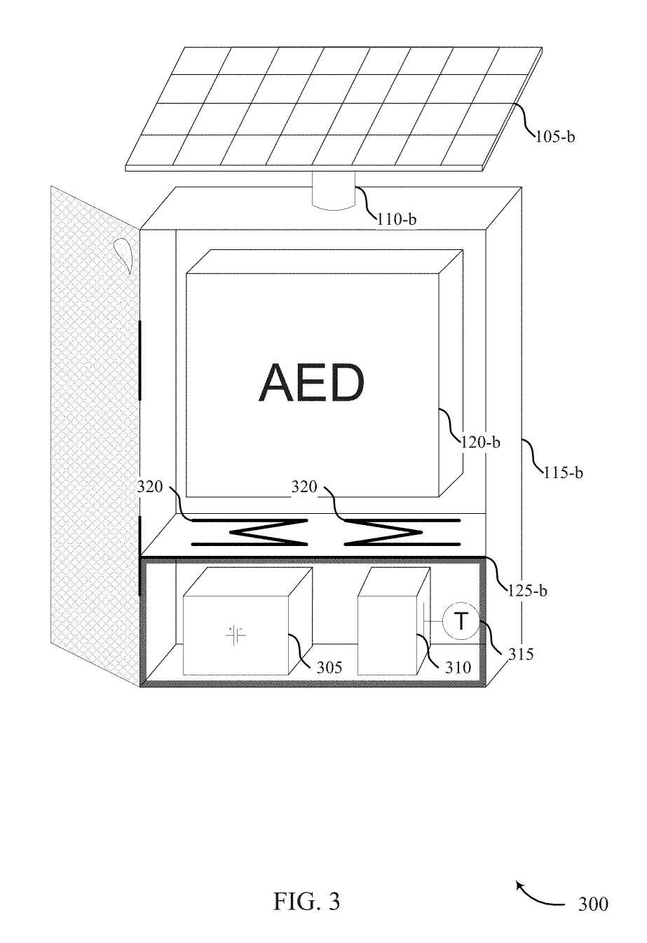

The enclosure 115 may include a mount 110 to which a solar panel 105 may be coupled. This solar panel 105 may provide power to a battery positioned within the enclosure 115 (described in more detail below with respect to FIG. 3), in order to power one or more heating elements configured to heat the enclosure 115 and ensure that the AED 120 is maintained at an operable temperature. The weatherproof nature of the enclosure 115 and the presence of the solar panel 105 may allow the enclosure 115 to be entirely self-contained and portable. This is at least because the enclosure 115 may not require wired electricity or other wired connection with a building, structure, or power source to maintain charge on the battery and maintain the enclosure 115 at an operable temperature with respect to the AED 120. Instead, enclosure 115 may be easily positioned and repositioned in various remote outdoor locations, such as runs at ski resorts or in parks, without the need for a wired power source or other connection.

The solar panel 105 may be rotatably coupled with the enclosure 115 via mount 110, such that the solar panel 105 may be positioned to absorb the most sunlight. This may also allow the enclosure 115 to remain in an outdoor position year-round, without the need to reposition the enclosure 155 itself; but instead requiring only repositioning of the solar panel 115 to coincide with the shifting position of the sun.

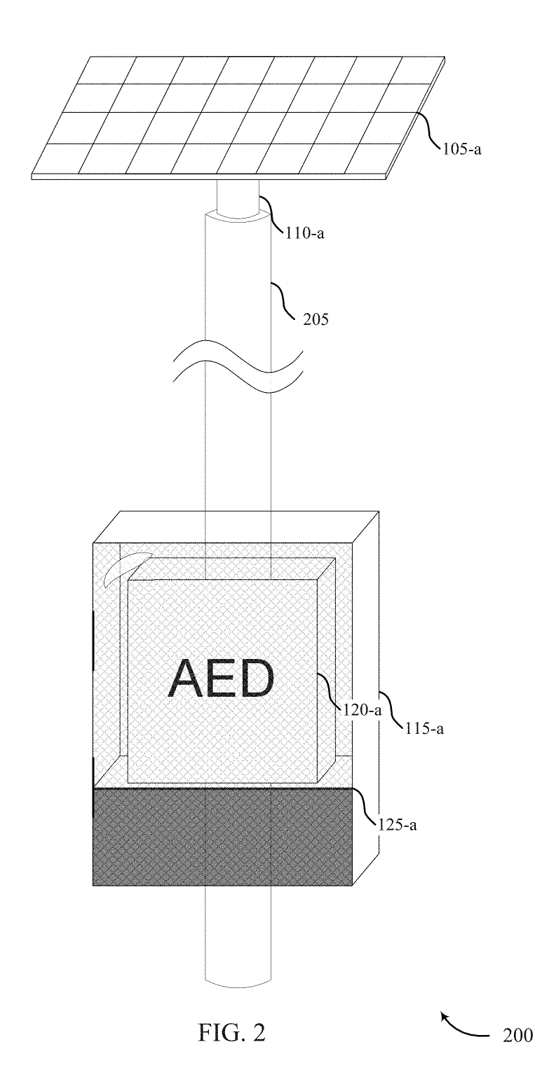

FIG. 2 is an example of an AED enclosure system 200 in accordance with various aspects of the disclosure. In some embodiments, the AED enclosure system 200 may include a solar panel 105-a, a mount 110-a, an enclosure 115-a, an AED 120-a, and/or a shelf 125-a, any of which may be examples of the corresponding components described above with respect to FIG. 1. The AED enclosure system 200 may further include a structure 205 to which the enclosure system 200 may be coupled. For example, structure 205 may be an example of any of a telephone pole, a sign, a fence post, or a light post, or any combination thereof. The enclosure system 200 may be permanently or removably coupled with the structure 205, such that the enclosure system 200 may be positioned in a convenient and easily accessible location. For example, structure 205 may be an example of a trail sign post in a park or wilderness area, and the enclosure system 200 may be coupled with the sign post structure 205 at a height that is easily accessible to hikers or other users in the area in need of urgent on-site defibrillation.

As shown in this illustration of enclosure system 200, a solar panel 105-a may be positioned atop the structure 205, so as to absorb maximum sunlight. In other examples, the solar panel 105-a may instead or additionally be coupled directly with the enclosure 115-a. In any example, the solar panel 105-a may be rotatably adjusted to face a direction most likely to result in maximum sunlight exposure.

FIG. 3 is an example of an interior view of an AED enclosure system 300 in accordance with various aspects of the disclosure. In some embodiments, the AED enclosure system 300 may include a solar panel 105-b, a mount 110-b, an enclosure 115-b, an AED 120-b, and/or a shelf 125-b, any of which may be examples of the corresponding components described above with respect to FIGS. 1 and 2. The enclosure system 300 may further include any of a battery 305, a controller 310, a thermostat 315, and/or one or more heating elements 320.

As previously discussed with respect to FIG. 1, the enclosure 115-b may include a front door or panel that may be easily removed or opened to provide easy user access to the AED 120-b positioned on shelf 125-b inside the enclosure 115-b. The AED 120-b may be any known, commercially available AED system, which may be self-contained and may be powered by a lithium-ion or other suitable battery. As previously discussed, the operability of the AED battery may be limited or entirely eliminated at lower temperatures, and it may therefore be useful to provide a consistently warmer climate within the enclosure 115-b, in order to regulate the temperature of the battery powering the AED 120-b. As illustrated in FIG. 1, enclosure 115-b may be heated by one or more heating elements 320. Heating elements 320 may be positioned above or below shelf 125-b, or in some examples may be integrated with shelf 125-b, and may be any known and commercially available heating modules, for example including metallic heating wires or coils surrounded by non-electrically conductive ceramic insulation. In any embodiment, the one or more heating elements 320 may be any self-contained heating element configured to conduct enough heat to increase and maintain the temperature of the interior of the enclosure 115-b.

In order to regulate the temperature of the enclosure 115-b, a thermostat 315 and controller 310 may operate in concert to detect the internal temperature of the enclosure 115-b and activate the one or more heating elements 320 upon detecting that the internal temperature of the enclosure 115-b has dropped below a predetermined first threshold temperature, such as 32 degrees Fahrenheit. Other predetermined first threshold temperatures, selected to coincide with the particular operating parameters of the AED 120-b battery, are also envisioned. A battery 305, for example a 12-volt lead-acid battery, may be positioned below the shelf 125-b in enclosure 115-b to provide power to the controller 310, thermostat 315, and one or more heating elements 320. As the one or more heating elements 320 operate to raise or maintain the temperature in the enclosure 115-b, the thermostat 315 may monitor the temperature in the enclosure 115-b continuously or at predetermined intervals. In some examples, the controller 310 may deactivate the one or more heating elements 320 upon detecting, via the thermostat 315, that the internal temperature of the enclosure 115-b has risen above the predetermined first threshold temperature. The thermostat 315 may then monitor the internal temperature of the enclosure 115-b continuously or at predetermined intervals while the one or more heating elements 320 are deactivated. Upon detecting that the internal temperature of the enclosure 115-b has again dropped below the predetermined first threshold temperature, the controller 310 may reactivate the one or more heating elements 320.

In some examples, in order to conserve energy, a predetermined second threshold temperature may be utilized to indicate that less frequent monitoring is required. For example, thermostat 315 may detect that the internal temperature of the enclosure 115-b has increased above a predetermined second threshold temperature, such as 70 degrees Fahrenheit. This may indicate that the AED enclosure system 300 is positioned in a warmer climate, or that the season is currently summer, such that, in either case, the internal temperature of the enclosure 115-b is unlikely to drop below the predetermined first threshold temperature. Based on detecting this predetermined second threshold temperature via thermostat 315, the controller 305 may accordingly deactivate the one or more heating elements 320, and may transition the thermostat 315 and the controller 305 to a low activity mode, such that the thermostat 315 may monitor the internal temperature of the enclosure 115-b at less frequent intervals. For example, the thermostat 315 may monitor the internal temperature of the enclosure 115-b at intervals of 10 minutes during normal operation, but may monitor the internal temperature of the enclosure 115-b at intervals of three hours during low activity mode operation. Other interval times appropriate to monitor and maintain the temperature of the enclosure 115-b are also envisioned.

In some examples, the AED enclosure system 300 may further include a global positioning system tracker positioned in, on, or about the enclosure 115-b. This global positioning system tracker may be communicatively coupled with a remote monitoring station, such that, upon detecting that the enclosure 115-b has been opened or that the AED 120-b has been activated, the remote monitoring station may receive a notification and location of the accessed or activated AED 120-b. In some examples, the remote monitoring station may use this information to send medical support to the location at which the AED 120-b was accessed, or may simply use the location information for recordkeeping purposes. In still other examples, the remote monitoring station may use location information received from the AED enclosure system 300 to detect that the AED enclosure system 300 has been moved, for example due to routine maintenance, or potentially due to theft. The remote monitoring station may accordingly use signals received from the global positioning system tracker to track the location of the AED enclosure system 300.

In some examples, the AED enclosure system 300 may additionally or alternatively include one or more tamper resistant features, for example to prevent children or animals from accessing the AED 120-b and potentially damaging the AED 120-b or harming themselves. Such tamper resistant features may include latches, locks, or the like on the enclosure 115-b to hinder access to the enclosure 115-b. In any embodiment, however, these tamper resistant features should not unduly hinder access to the AED 120-b for users experiencing a life threatening cardiac event.

Having described several embodiments, it will be recognized by those of skill in the art that various modifications, alternative constructions, and equivalents may be used without departing from the spirit of the invention. For example, the above elements may merely be a component of a larger system, wherein other rules may take precedence over or otherwise modify the application of the invention. Also, a number of steps may be undertaken before, during, or after the above elements are considered. Accordingly, the above description should not be taken as limiting the scope of the invention.

What is claimed is:

1. A system for storing an automated external defibrillator (AED), comprising:
an enclosure comprising a first portion to store the AED, and a second portion to store a battery to power a heating element;
a thermostat to monitor a temperature of the enclosure, the thermostat contained within the second portion of the enclosure;
the heating element to heat the first portion of the enclosure, the heating element in electrical communication with the thermostat and contained within the first portion of the enclosure; and
a controller to activate and deactivate power to the heating element, the controller in electrical communication with the battery and the heating element, and contained within the second portion of the enclosure.

2. The system of claim 1, further comprising a solar panel in electrical communication with the battery.

3. The system of claim 2, wherein the solar panel is positioned remotely from the enclosure containing the AED.

4. The system of claim 3, wherein the solar panel is positioned atop a structure, and wherein the enclosure containing the AED is affixed at a base of the structure.

5. The system of claim 4, wherein the structure is any of a telephone pole, a sign, a fence post, or a light post, or a combination thereof.

6. The system of claim 1, wherein the enclosure is constructed of a rigid, waterproof material.

7. The system of claim 1, wherein the battery is a 12V lead-acid battery.

8. The system of claim 1, wherein the enclosure further comprises:
a global positioning system tracker or tamper resistant features, or a combination thereof.

9. The system of claim 1, wherein the controller is operable to activate power to the heating element when the temperature of the second portion of the enclosure falls below 32° F.

10. An enclosure for an automated external defibrillator (AED), comprising:
a first portion of the enclosure configured for storing the AED, wherein the first portion of the enclosure comprises:
means for heating the first portion of the enclosure;
a second portion of the enclosure separate from the first portion and configured for storing a battery to power the means for heating the first portion of the enclosure, wherein the second portion of the enclosure comprises:
means for monitoring a temperature of the second portion of the enclosure;
means for determining that the temperature of the second portion of the enclosure is below a first threshold; and
means for initiating the means for heating based at least in part on determining that the temperature of the second portion of the enclosure is below the first threshold.

11. The enclosure of claim 10, wherein:
the means for determining that the temperature of the second portion of the enclosure is below the first threshold further comprises means for determining that the temperature of the second portion of the enclosure is above a second threshold; and
the means for initiating the means for heating comprises means for deactivating the means for heating based, at least in part, on determining that the temperature of the second portion of the enclosure is above the second threshold.

12. The enclosure of claim 10, further comprising means for powering the means for initiating heating of the enclosure.

13. the enclosure of claim 12, wherein the means for powering the means for initiating heating of the enclosure is configured to rotate or separate from the enclosure, or a combination thereof.

14. The enclosure of claim 13, wherein the means for powering the means for initiating the heating of the enclosure is positioned atop a structure, and wherein the enclosure for the AED is positioned at the base of the structure.

15. The enclosure of claim 14, wherein the structure is at least one of a telephone pole, a sign, a fence post, or a light post, or any combination thereof.

16. The enclosure of claim 10, wherein the enclosure is configured to be portable, or waterproof, or a combination thereof.

17. The enclosure of claim 10, wherein the enclosure comprises:
means for tracking the position of the enclosure or providing tamper resistant features on the enclosure, or a combination thereof.

18. The enclosure of claim 10, wherein the first threshold comprises 32° F.

19. A system for heating a battery to power an automated external defibrillator (AED), comprising:
an enclosure comprising a first portion to store the AED, and a second portion to store a battery to power a heating element, the first portion comprising a mounting bracket to support the AED;
a thermostat to monitor a temperature of the second portion of the enclosure, the thermostat contained within the second portion;
the heating element to heat the first portion of the enclosure and in electrical communication with the thermostat, and contained within the first portion of the enclosure;
a controller in electrical communication with the battery to power the heating element and the heating element, the controller contained within the second portion of the enclosure and operable to activate power to the heating element when the monitored temperature of the second portion drops below 32° F. and deactivate power to the heating element when the monitored temperature of the enclosure increases above a threshold; and
a solar panel to charge the battery to power the heating element, the solar panel affixed to an exterior portion of the enclosure and in electrical communication with the battery to power the heating element.

20. The system of claim 19, wherein the solar panel is positioned remotely from the enclosure comprising the AED.
